(19) United States
(12) Patent Application Publication
(10) Pub. No.: US 2026/0314780 A1
(43) Pub. Date: Oct. 15, 2026

(54) AMBULATORY CHEMOTHERAPY CONTAINMENT BAG

(71) Applicant: Lorna Brunelle, Middleborough, MA (US)

(72) Inventor: Lorna Brunelle, Middleborough, MA (US)

(21) Appl. No.: 19/648,522

(22) Filed: Apr. 15, 2026

Related U.S. Application Data
(60) Provisional application No. 63/788,850, filed on Apr. 15, 2025.

(51) Int. Cl. A45C 13/02; A45C 3/00; A45C 3/06; A45C 13/00; A45F 3/00; A45F 3/02; A61M 5/142

(52) U.S. Cl. CPC A45C 13/02; A45C 3/001; A45C 3/06; A45C 13/008; A45F 3/02; A61M 5/14244; A45F 2003/003

(57) ABSTRACT

An improved ambulatory chemotherapy containment bag is provided that is more convenient, includes safer containment both for the chemotherapy pouch and the delivery lines associated therewith, and includes sound proofing of the pump system contained therein.

### CROSS-REFERENCE TO RELATED APPLICATIONS

This application claims priority from U.S. Patent Application No. 63/788,850, filed Apr. 15, 2025.

### BACKGROUND OF THE INVENTION

The present disclosure relates to a device to provide an improved ambulatory chemotherapy containment bag. More specifically, the present disclosure relates to an improved ambulatory chemotherapy bag that provides better convenience, safer containment both for the chemotherapy pouch and the delivery lines associated therewith, and sound proofing of the pump system contained therein.

Cancer is one of the leading causes of death worldwide, contributing to approximately 7.6 million deaths per annum (around 13% of all deaths). The number of newly diagnosed cancer cases every year in the United States alone is over 2 million, of which over 50% of patients receive chemotherapy as their primary treatment method.

Conventional chemotherapy treatment often presents complications, such as adverse physical side effects, and is an intrusive treatment delivery method, resulting not only in physical discomfort for the patient but also psychological distress. These issues considerably reduce a patient's ability to carry out daily activities, commonly resulting in reduced independence, which in turn impinges on their recovery rates. Furthermore, the primary side effect experienced by patients receiving chemotherapy is that their immune response is suppressed, which makes them susceptible to infection. Studies have found that infection is the primary cause of death for patients receiving chemotherapy treatment (eliminating disease progression fatalities).

Research has revealed that the use of a continuous low dose infusion of the chemotherapy drugs has proven to alleviate the side effects experienced during the treatment. The slow infusion method increases the time in which the drug is administered from the current 4-6 hour intensive treatment to a 96-hour infusion cycle. In addition, it has been observed that the slow infusion method has generated a greater cellular response to the chemotherapy drugs, resulting in a more effective treatment method.

Portable infusion pump devices may be used to deliver the chemotherapy drugs. Common complaints about current portable infusion pump devices vary from noise of the machine to the inability to comfortably wear the product during the day when carrying out daily tasks. Due to the noise and uncomfortable or clumsy product configuration of many current pump devices, sleeping with the device is undesirable. Patients have reported that the portable pump has improved their attitudes towards the treatment as it has provided them with greater autonomy; however, the current pump designs limit the portability and comfort of such pumps due to their large size. In addition, these existing devices weigh an average of 700 grams including drug fluid, further hindering patient comfort.

The inability to wear these products comfortably whilst sleeping, results in a limitation on the time the chemotherapy drugs can be delivered into the body, consequently not maximizing the full potential of the continuous low dose release system.

It is a goal of the present disclosure to address or ameliorate one or more shortcomings or disadvantages of prior intravenous fluid delivery techniques, or to at least provide a useful alternative thereto.

### SUMMARY OF THE INVENTION

In accordance with the present disclosure an improved ambulatory chemotherapy containment bag is provided. More specifically, an improved ambulatory chemotherapy bag is disclosed that provides better convenience, safer containment both for the chemotherapy pouch and the delivery lines associated therewith, and sound proofing of the pump system contained therein.

As stated above, it is preferable to provide portable infusion pump devices for delivery of chemotherapy drugs to patients. In this manner, rather than using an intensive 4-6 hour intensive treatment for the delivery of the drugs, a slow infusion method increases the time in which the drug is administered to a 96-hour infusion cycle. To address one of the common complaints about current portable infusion pump devices the present disclosure provides a portable carrier that is more comfortable and convenient as compared to the carry systems of the prior art.

In some embodiments, an ambulatory chemotherapy bag in accordance with the current disclosure employs softer materials for construction of the outer surfaces thereof in contrast to the ballistic nylon materials typically used. This provides a softer touch and feel for the user. Further, the strap arrangement for carrying and securing the bag to the patient employs wider and softer strap materials with improved swivels and connectors to allow the strap higher a freedom of movement as the bag is being carried by the patient. These improvements allow a user to comfortably wear the product during the day while carrying out daily tasks.

In other embodiments, the bag is designed with multiple compartments therein that are segregated from one another and specifically designed. Chemotherapy drugs are highly toxic to both humans and pets. Accordingly, one compartment of the bag is configured and arranged to contain the bag that holds the therapeutic agent itself. This compartment is lined with an abrasion resistant material that is fully waterproofed and configured and arranged to contain spills and/or leaks. The liner compartment includes a lid that can be secured when the therapeutic agent is contained therein thereby protecting the therapeutic from exposure to light while fully containing the therapeutic agent in the event of a rupture of spill. In this manner, the therapeutic agent is fully shielded from accidental contact with users, people that come into contact with the user, pets, etc. This liner may be removable from its designated compartment within the bag for cleaning and the like.

In a further embodiment, a compartment is provided with a liner for containing the intravenous pump itself. This compartment is preferably formed to include the same features as the therapeutic agent compartment with shielding against leaks etc., but further includes a layer of soundproofing material. Due to the noise and uncomfortable or clumsy product configuration of many current pump devices, sleeping with prior art devices is undesirable. The soundproofing material contains the noise of pump operation thereby making the portable pump more comfortable for the user while maintaining a quiet environment for user sleep.

Additional compartments are provided in the bag to serve as convenient storage of items such as keys, wallets, snacks, etc. It should be appreciated that due to the shielding of the therapeutic agent and pump as described above, items stored in these compartments are kept safe from accidental spills and/or leaks.

In some embodiments, the tubing set/IV lines required to deliver the therapeutic agent from the pump to the user are also shielded to prevent abrasion. In particular, the shielding prevents accidental ruptures of the tubing through snags, abrasion, and even from pets chewing on them. Such shielding is preferably abrasion resistant material. Further, such shielding is preferable reinforced material such as aramid fiber or carbon fiber. Further, the shielding provides further protection against leaks and/or accidental exposure of children and/or pets, which could be fatal.

In addition to the features above, the bag of the present disclosure may be ornamented or decorated in a manner that causes it to look more like a conventional tote or handbag. This makes it more attractive for users and conceals its real purpose thereby increasing the comfort of the wearer.

It is therefore an object of the present disclosure to provide an improved ambulatory chemotherapy containment bag. It is a further object of the present disclosure to provide an improved ambulatory chemotherapy bag that facilitates better convenience, safer containment both for the chemotherapy pouch and the delivery lines associated therewith, and sound proofing of the pump system contained therein.

These together with other objects of the disclosure, along with various features of novelty, which characterize the method and system, are pointed out with particularity in the claims annexed hereto and forming a part of this disclosure. For a better understanding of the method and system, its operating advantages and the specific objects attained by its uses, reference should be had to the accompanying drawings and descriptive matter in which there is illustrated a preferred embodiment of the method and system.

### BRIEF DESCRIPTION OF THE DRAWINGS

In the drawings which illustrate the best mode presently contemplated for carrying out the present invention:

FIG. 1 is a perspective view of an ambulatory chemotherapy containment bag in accordance with an embodiment of the present disclosure.

FIG. 2 is a front elevational view of the bag of FIG. 1, illustrating an exterior configuration and strap assembly.

FIG. 3 is a cross-sectional view taken along the line 3-3 of the bag of FIG. 2, illustrating internal compartments including a therapeutic agent compartment and an infusion pump compartment.

FIG. 4 is a detailed view of a tubing pathway and sealed port extending through a wall of the bag.

FIG. 5 is a detailed view of a protective sheath and reinforced tubing conduit associated with the bag.

FIG. 6 is an exploded view of a fluid-impermeable liner configured for use within the therapeutic agent compartment.

FIG. 7 is a perspective view illustrating a strap assembly including a multi-axis swivel connector.

### DETAILED DESCRIPTION OF THE DISCLOSURE

Now referring to the drawings, an improved ambulatory chemotherapy containment bag is provided. More specifically, the disclosure is directed to a portable chemotherapy containment system that enhances user comfort while providing improved safety in containing a therapeutic agent reservoir, associated delivery tubing, and an infusion pump. The system further provides sound attenuation for the pump and structural protection for fluid pathways, thereby enabling extended ambulatory use.

Portable infusion pump devices are commonly used to deliver chemotherapy drugs over extended durations. Rather than requiring relatively short, high-intensity treatment sessions of approximately 4-6 hours, such devices enable slow infusion protocols extending up to approximately 96 hours or longer. However, existing carrying solutions are often uncomfortable, noisy, and insufficiently protective against leaks or tubing damage. The present disclosure addresses these shortcomings by providing a structurally integrated carrier system that combines containment, protection, and ergonomic wearability.

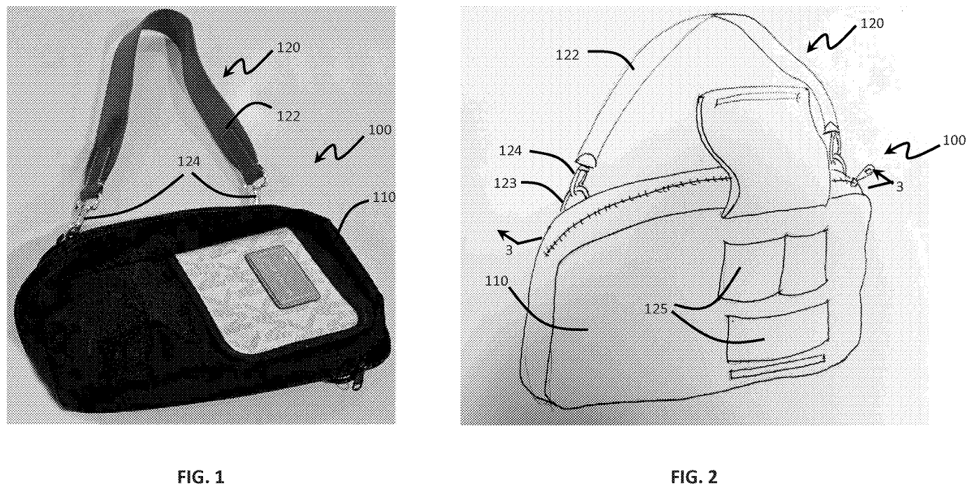

Referring to FIG. 1, an ambulatory chemotherapy containment bag 100 is shown. The bag 100 includes a body 110 defining an interior volume. The body 110 may be formed from a flexible, compliant material such as woven fabric, synthetic polymer textiles, or composite materials. In certain embodiments, the exterior surface of the body 110 is formed from a soft, compliant material that provides enhanced tactile comfort relative to conventional ballistic nylon constructions.

The body 110 may further include structural panels or semi-rigid inserts that maintain the shape of the bag while allowing flexibility for user movement. The overall form factor may resemble a conventional tote, handbag, or shoulder bag, thereby reducing the visual appearance of a medical device and improving user acceptance and discretion.

As shown in FIGS. 1 and 2, the bag 100 includes a strap assembly 120 configured to facilitate carrying by a user. The strap assembly 120 may include one or more padded straps 122 and connectors 124 that engage the bag at strap attachment points 123.

In certain embodiments, the strap 122 includes padding and has a width greater than approximately 2 inches to distribute load and improve comfort during extended wear. The connectors 124 may be configured as swivel connectors permitting multi-axis movement, thereby allowing the bag 100 to move naturally with the user's body and reducing strain.

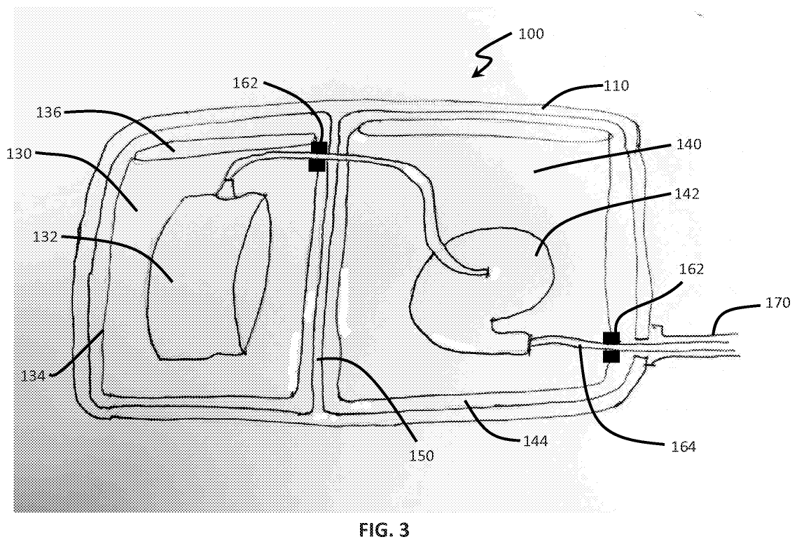

Referring to FIG. 3, a first compartment 130 is disposed within the body 110 and is configured to receive a therapeutic agent reservoir 132, such as a chemotherapy drug pouch or container. The first compartment 130 includes a fluid-impermeable liner 134 configured to contain leaks or spills of the therapeutic agent.

As shown in FIG. 6, the liner 134 may comprise a multi-layer structure including an inner polymeric barrier layer 134a, such as thermoplastic polyurethane (TPU) or polyethylene, and an outer abrasion-resistant layer 134b configured to resist puncture and wear.

The first compartment 130 further includes a closable cover 136, which may be implemented as a zipper, flap, roll-top closure, or other sealing mechanism. In some embodiments, the closure 136 forms a substantially sealed enclosure that limits fluid escape in the event of rupture of the therapeutic reservoir 132.

In certain embodiments, the first compartment 130 includes light-blocking material configured to reduce exposure of the therapeutic agent to ambient light, thereby preserving drug stability.

The liner 134 may be removably secured within the first compartment 130 using fasteners such as hook-and-loop elements, snaps, zippers, or mechanical clips, thereby facilitating cleaning and replacement.

Also shown in FIG. 3, a second compartment 140 is disposed within the body 110 and is configured to receive an infusion pump 142. The second compartment 140 is physically separated from the first compartment 130.

The second compartment 140 includes a sound-dampening layer 144 configured to reduce operational noise generated by the infusion pump 142. The sound-dampening layer 144 may comprise a foam material such as closed-cell foam, open-cell acoustic foam, or viscoelastic foam acoustic absorption layer positioned about at least a portion of the infusion pump and configured to attenuate sound transmission generated during pump operation.

In certain embodiments, the sound-dampening layer 144 is arranged to substantially surround the infusion pump 142, forming a partial or complete enclosure that attenuates sound transmission. The layer may be integrated into the compartment walls or provided as a removable insert.

As further illustrated in FIG. 3, the first compartment 130 and the second compartment 140 are structurally isolated from one another to limit fluid communication therebetween.

This isolation may be achieved using a barrier wall 150 disposed between the compartments. The barrier wall 150 may comprise a fluid-resistant material, such as a polymer-coated fabric or laminated composite, and may optionally include a sound-attenuating layer.

The structural isolation ensures that leakage occurring within the first compartment 130 is contained therein and does not contaminate the second compartment 140 or other portions of the bag 100.

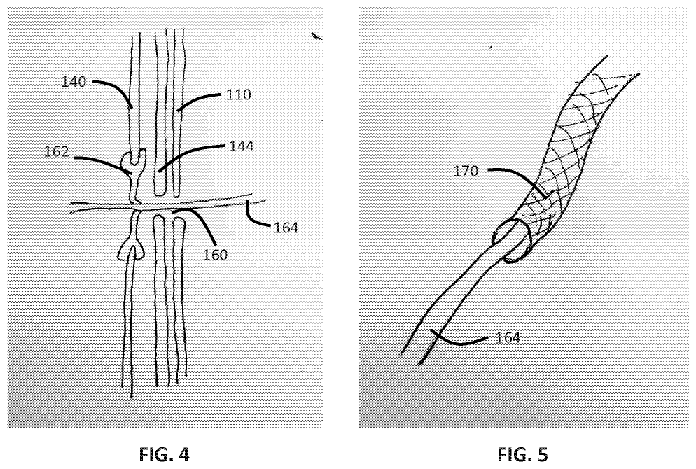

Referring to FIG. 4, the bag 100 includes a tubing pathway 160 extending from at least one of the first compartment 130 or the second compartment 140 to an exterior of the body 110. The tubing pathway 160 provides a controlled route for intravenous tubing 164.

The tubing pathway 160 may include a sealed port 162 extending through a wall of the body 110. The sealed port 162 may include flexible grommets, elastomeric seals, or compression fittings configured to reduce fluid leakage and protect the tubing 164 from wear.

In certain embodiments, the tubing pathway 160 forms part of a dedicated routing channel integrated into the structure or seams of the bag 100, thereby maintaining tubing alignment and reducing kinking or snagging.

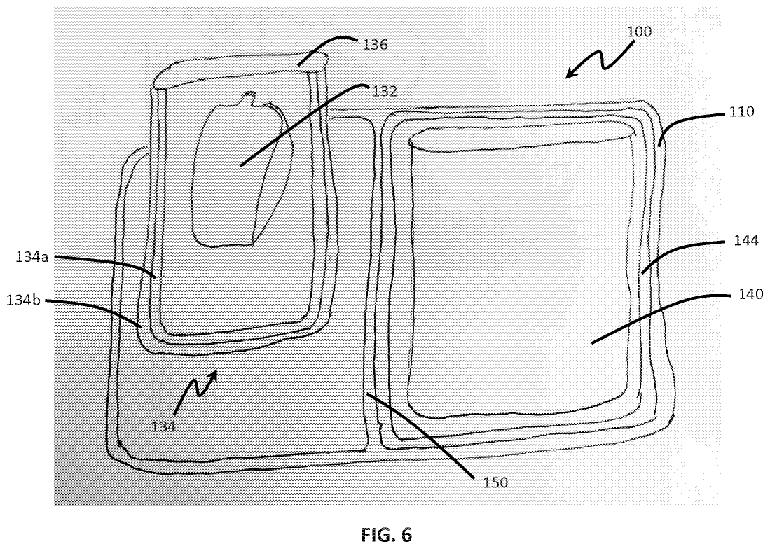

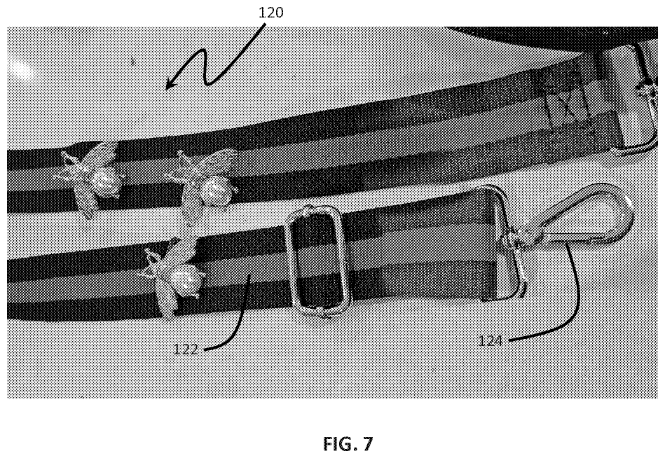

Referring to FIG. 5, a protective sheath 170 is associated with the tubing 164 and is configured to protect the tubing from external damage. The protective sheath 170 comprises an abrasion-resistant material.

In certain embodiments, the protective sheath 170 comprises a multi-layer composite structure including an inner flexible layer and a reinforcement layer. The reinforcement layer may include materials such as aramid fibers, carbon fiber composites, or combinations thereof.

The protective sheath 170 may be removably attachable to the bag 100 using connectors such as zippers, clips, or modular fasteners. The sheath 170 is configured to resist external interference, including abrasion, snagging, and animal interaction, thereby reducing the risk of tubing rupture.

The bag 100 as shown at FIG. 2, may further include one or more auxiliary compartments 125 configured to store personal items such as keys, wallets, or mobile devices. These auxiliary compartments are isolated from the first compartment 130 such that stored items remain protected from contamination in the event of leakage.

In addition to the functional features described above, the bag 100 may be configured to resemble a non-medical personal accessory, such as a handbag, tote, or messenger bag. This improves user discretion and comfort by concealing the medical nature of the device.

The combination of structural isolation, fluid containment, sound attenuation, and tubing protection provides a comprehensive ambulatory infusion system that improves safety, usability, and patient quality of life during extended treatment periods.

Accordingly, the present disclosure provides an improved ambulatory chemotherapy containment bag that integrates multiple protective and ergonomic features into a unified system. While specific embodiments have been described with reference to the accompanying drawings, it will be appreciated that various modifications and alternative configurations may be employed without departing from the scope of the appended claims. It can therefore be seen that the present disclosure provides an improved ambulatory chemotherapy containment bag is provided that is more convenient, includes safer containment both for the chemotherapy pouch and the delivery lines associated therewith, and includes sound proofing of the pump system contained therein. For these reasons, the present disclosure is believed to represent a significant advancement in the art, which has substantial commercial merit.

While there is shown and described herein certain specific structure embodying the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

What is claimed:

1. An ambulatory chemotherapy containment bag, comprising:
a body defining an interior;
a first compartment disposed within the body and configured to receive a therapeutic agent reservoir, the first compartment including a fluid-impermeable liner and a closable cover forming a sealed containment barrier configured to retain leaked therapeutic fluid within the first compartment;
a second compartment disposed within the body and physically separated from the first compartment, the second compartment configured to receive an infusion pump and including a sound-dampening layer;
a tubing pathway extending from at least one of the first compartment or the second compartment to an exterior of the body; and
a protective sheath associated with the tubing pathway, the protective sheath comprising an abrasion-resistant material configured to resist abrasion, snagging, and external compression forces acting on the tubing during ambulatory use,
wherein the first compartment and the second compartment are structurally isolated from one another to limit fluid communication therebetween.

2. The bag of claim 1, wherein the fluid-impermeable liner comprises a multi-layer structure including an inner polymeric barrier layer and an outer abrasion-resistant layer.

3. The bag of claim 1, wherein the first compartment further includes light-blocking material configured to reduce exposure of the therapeutic agent to ambient light.

4. The bag of claim 1, wherein the fluid-impermeable liner is removably secured within the first compartment.

5. The bag of claim 1, wherein the sound-dampening layer comprises a viscoelastic or cellular acoustic absorption layer positioned about at least a portion of the infusion pump and configured to attenuate sound transmission generated during pump operation.

6. The bag of claim 5, wherein the sound-dampening layer is arranged to substantially surround the infusion pump.

7. The bag of claim 1, wherein the tubing pathway includes a sealed port extending through a wall of the bag.

8. The bag of claim 1, wherein the protective sheath is removably attachable to the bag.

9. The bag of claim 1, wherein the protective sheath comprises a multi-layer composite structure.

10. The bag of claim 1, further comprising one or more auxiliary compartments configured to store personal items, the auxiliary compartments being isolated from the first compartment.

11. The bag of claim 1, wherein the body comprises an outer surface formed from a compliant material providing enhanced user comfort relative to ballistic nylon.

12. The bag of claim 1, wherein the first compartment and the second compartment are separated by a barrier wall comprising a fluid-resistant material.

13. The bag of claim 1, wherein the bag body is configured to resemble a non-medical personal accessory.

14. A portable infusion carrier system, comprising:
a bag body;
a first sealed compartment configured to contain a chemotherapy reservoir and including a removable, fluid-impermeable liner;
a second compartment configured to contain an infusion pump, the second compartment including a noise-reducing material layer;
a strap assembly coupled to the bag body, the strap assembly including at least one flexible connector providing multi-axis movement; and
a reinforced conduit configured to receive an intravenous line extending from the bag body,
wherein the first sealed compartment is configured to contain leaks independent of the second compartment.

15. The bag of claim 14, wherein the strap assembly includes a padded strap having a width greater than 2 inches.

16. The bag of claim 14, wherein the flexible connector comprises a swivel connector permitting rotation about at least two axes.

17. The system of claim 14, wherein the reinforced conduit further includes a protective outer sleeve configured to prevent animal interference.

18. An ambulatory infusion apparatus, comprising:
a portable bag;
an infusion pump disposed within the bag;
a therapeutic fluid reservoir disposed within the bag; and
a tubing management system comprising:
a dedicated routing channel extending through a wall of the bag; and
a reinforced sheath surrounding at least a portion of tubing extending through the routing channel,
wherein the reinforced sheath comprises a material selected from aramid fibers, carbon fiber composites, or combinations thereof, and is configured to resist abrasion and external interference.
